(19) United States
(12) Patent Application Publication
(10) Pub. No.: US 2004/0010175 A1
(43) Pub. Date: Jan. 15, 2004

(54) Catalyst and process for contacting a hydrocarbon and ethylene

(76) Inventors: Bruce B. Randolph, Bartlesville, OK (US); James B. Kimble, Bartlesville, OK (US); Marvin M. Johnson, Bartlesville, OK (US)

Correspondence Address: RICHMOND, HITCHCOCK,, FISH & DOLLAR, P.O. Box 2443, Bartlesville, OK, 74005, US

(21) Appl. No.: 10/193,058

(22) Filed: Jul. 11, 2002

(51) Int. Cl. C07C 2/56; C07C 6/08; C07C 5/13

(52) U.S. Cl. 585/708; 585/709; 585/734

(57) ABSTRACT

A process of contacting at least one feed hydrocarbon, containing three to about seven carbon atoms per molecule, and ethylene in a hydrocarbon-containing fluid in the presence of a catalyst composition to provide at least one product hydrocarbon isomer containing about four to about nine carbon atoms per molecule is provided. The at least one feed hydrocarbon can be selected from paraffins, isoparaffins, and the like and combinations thereof. The catalyst composition contains a hydrogen halide component, a sulfone component, and a metal halide component.

### BACKGROUND OF THE INVENTION

The present invention relates to a process of contacting a hydrocarbon and ethylene in the presence of a catalyst composition.

Oxidative coupling of methane is well known to produce a product mixture containing, among other components, ethylene, ethane, propane, and propylene. For many applications of this technology, higher molecular weight products are necessary. In such applications, a second conversion is typically required. The process of conducting the second conversion usually requires a commercially available olefin-to-gasoline process. Such process typically employs a zeolitic material such as ZSM-5 to accomplish the oligomerization of ethylene and propylene to higher molecular weight materials. However, ZSM-5 is well known to coke rapidly under the reaction conditions required for such conversion. The process required to accommodate the tendency of such catalyst material to rapidly coke is difficult and expensive. Thus, a process of contacting a hydrocarbon, such as a paraffin, and ethylene to produce higher molecular weight material without the need for the use of a zeolitic material such as ZSM-5 would be a significant contribution to the art and to the economy.

Further, processes of alkylating an isoparaffin such as isobutane with an olefin containing from three to five carbon atoms per molecule and the disproportionation of isopentane with catalysts comprising hydrofluoric acid, sulfolane, and TiF₄ are known. However, such catalyst systems have not been effectively employed for the converting paraffins, such as normal paraffins, with ethylene at moderate reaction conditions. Thus, a process of converting a paraffin, such as a normal paraffin, with ethylene utilizing a catalyst system at moderate reaction conditions that does not require the use of a zeolitic material would also be of significant contribution to the art and to the economy.

### SUMMARY OF THE INVENTION

It is an object of the present invention to provide a process for contacting a hydrocarbon selected from the group consisting of paraffins (also referred to as alkanes), isoparaffins (also referred to as isoalkanes), and the like and combinations thereof containing from about three to about seven carbon atoms per molecule and ethylene in the presence of a catalyst composition under conversion conditions to provide at least one product hydrocarbon isomer comprising an isoparaffin containing from about four to about nine carbon atoms per molecule. The process can be utilized at moderate conversion conditions without the need for separate steps or separate conversions utilizing zeolitic materials such as ZSM-5.

Another object of the present invention is to provide a process that comprises contacting a hydrocarbon selected from the group consisting of paraffins, isoparaffins and the like and combinations thereof containing from about three to about seven carbon atoms per molecule and ethylene to provide higher molecular weight materials such as isoparaffins containing from about four to about nine carbon atoms per molecule.

Another object of the present invention is to provide a process that comprises contacting an initial isoparaffin containing from about four to about five carbon atoms per molecule and ethylene to provide an isoparaffin having a higher number of carbon atoms per molecule than the initial isoparaffin.

An embodiment of the present invention comprises a process comprising contacting at least one feed hydrocarbon selected from the group consisting of paraffins, isoparaffins, and the like and combinations thereof containing from about three to about seven carbon atoms per molecule and ethylene in the presence of a catalyst composition under conversion conditions to provide at least one product hydrocarbon isomer comprising an isoparaffin containing from about four to about nine carbon atoms per molecule. A catalyst composition of the present invention comprises a hydrogen halide component, a sulfone component, and a metal halide component. Such a process utilizes moderate conversion conditions and can be adapted to include additional hydrocarbon reactions such as alkylation, isomerization, disproportionation, and the like and combinations thereof.

Another embodiment of the present invention comprises a process comprising contacting at least one feed hydrocarbon selected from the group consisting of paraffins, isoparaffins, and the like and combinations thereof containing from about three to about seven carbon atoms per molecule and ethylene in the presence of a catalyst composition under conversion conditions to provide at least one product hydrocarbon isomer comprising an isoparaffin having a higher number of carbon atoms per molecule than the initial hydrocarbon that is converted. A catalyst composition of the present invention comprises a hydrogen halide component, a sulfone component, and a metal halide component. Such a process utilizes moderate conversion conditions and can be adapted to include additional hydrocarbon conversion reactions such as alkylation, isomerization, disproportionation, and the like and combinations thereof.

Other objects and advantages of the present invention will become apparent from the detailed description and the appended claims.

### DETAILED DESCRIPTION OF THE INVENTION

It has been discovered that a hydrocarbon selected from the group consisting of paraffins (also referred to as alkanes), isoparaffins (also referred to as isoalkanes), and the like and combinations thereof comprising from about three to about seven carbon atoms can be contacted with ethylene in the presence of a catalyst composition under conversion conditions to provide an isoparaffin comprising from about four to about nine carbon atoms per molecule where such catalyst composition comprises a hydrogen halide component, a sulfone component, and a metal halide component.

Generally, a conversion process, such as an alkylation process, involves the catalytic alkylation of olefins, also referred to as alkenes, with isoparaffins. Generally, alkylation processes are liquid phase processes wherein olefins such as propylene, butylenes, pentylenes, hexylenes, heptylenes, octylenes, and the like are alkylated by an isoparaffin hydrocarbon such as isobutane, isopentane, isohexane, isoheptane, isooctane and the like for production of high octane alkylate hydrocarbons boiling in the gasoline range and which are suitable for use in a gasoline motor fuel. A novel and inventive aspect of the present invention is that such typical alkylation processes and reactor designs can now be utilized with minimal design modifications to contact hydrocarbons such as paraffins and ethylene to provide isoparaffins by utilizing a novel process of utilizing a catalyst composition comprising a hydrogen halide component, a sulfone component, and a metal halide component.

Paraffins that can be utilized in a process of the present invention include any paraffin that can be contacted with ethylene according to a process of the present invention. Examples of suitable paraffins include, but are not limited to, paraffins containing from about three to about seven carbon atoms per molecule, preferably containing from about three to about five carbon atoms per molecule. Generally, the paraffins comprise normal paraffins including, but not limited to, propane, butane, pentane, hexane, heptane, and the like and combinations thereof. Preferably, the paraffins comprise normal paraffins including, but not limited to, propane, butane, pentane, and the like and combinations thereof. More preferably, the paraffins comprise propane or butane. Most preferably, the paraffins comprise butane.

Isoparaffins, also referred to as isoalkanes, that can be provided utilizing a process of the present invention include isoparaffins containing from about four to about nine carbon atoms per molecule. The isoparaffins provided by a process of the present invention typically have a higher molecular weight than the hydrocarbons selected from the group consisting of paraffins, isoparaffins, and the like and combinations thereof that are contacted with ethylene according to a process of the present invention. Examples of suitable isoparaffins that can be provided by a process of present invention include, but are not limited to, isobutane, isopentane, 2,2-dimethylbutane, 2,3-dimethylbutane, 2-methylpentane, 3-methylpentane, 2,2-dimethylpentane, 2,4-dimethylpentane, 2,2,3-trimethylbutane, 3,3-dimethylpentane, 2-methylhexane, 2,3-dimethylpentane, 3-methylhexane, 3-ethylpentane, 2,2,4-trimethylpentane, 2,2-dimethylhexane, 2,5-dimethylhexane, 2,4-dimethylhexane, 3,3-dimethylhexane, 2,3,4-trimethylpentane, 2,3,3-trimethylpentane, 2,3-dimethylhexane, 2-methyl-3-ethylpentane, 2-methylheptane, 4-methylheptane, 3,4-dimethylhexane, 3-methylheptane, 2,2,5-trimethylhexane, and the like and combinations thereof. Preferably, an isoparaffin provided by a process of present invention comprises isobutane or isopentane. More preferably, an isoparaffin provided by a process of present invention comprises isobutane.

A process of the present invention can also comprise contacting an isoparaffin and ethylene in the presence of a catalyst composition of the present invention. When isoparaffins are contacted with ethylene according to a process of the present invention, the isoparaffins that are provided typically have a higher molecular weight or contain more carbon atoms per molecule than the isoparaffin that is contacted. For example, when an isoparaffin such as isopentane is contacted with ethylene according to a process of the present invention, the isoparaffin can be 2,3-dimethylbutane, 2-methylpentane, 3-methylpentane, and the like.

The isoparaffins that are can be initially present and contacted with ethylene utilizing a process of the present invention typically include those isoparaffins comprising from about four to about seven carbon atoms per molecule. Examples of suitable isoparaffins that can be initially present and contacted with ethylene utilizing a process of present invention include, but are not limited to, isobutane, isopentane, 2,2-dimethylbutane, 2,3-dimethylbutane, 2-methylpentane, 3-methylpentane, 2,2-dimethylpentane, 2,4-dimethylpentane, 2,2,3-trimethylbutane, 3,3-dimethylpentane, 2-methylhexane, 2,3-dimethylpentane, 3-methylhexane, 3-ethylpentane, and the like and combinations thereof. Preferably, an isoparaffin initially present and contacted with ethylene utilizing a process of the present invention comprises isobutane or isopentane. More preferably, an isoparaffin initially present and contacted with ethylene utilizing a process of the present invention comprises isobutane.

The term “feed hydrocarbon” as used herein refers to any hydrocarbon present in a hydrocarbon-containing fluid of the present invention that is contacted, preferably converted, with ethylene to provide an isoparaffin according to a process of the present invention. For example, at least one feed hydrocarbon can be a normal paraffin as described herein.

The term “fluid” as used herein refers to gas, liquid, vapor, and combinations thereof.

The term “product hydrocarbon isomer” as used herein refers to any hydrocarbon present in a product of a process of the present invention that has been provided by contacting a hydrocarbon and ethylene according to a process of the present invention.

Preferably, contacting at least one feed hydrocarbon and ethylene in the presence of a catalyst composition utilizing a process of the present invention provides for a converting of the at least one feed hydrocarbon. The term “converting” or “conversion” as used herein refers to any change in a hydrocarbon, including ethylene, as described herein as a result of utilizing a process of the present invention. Examples of suitable converting or conversion include, but are not limited to, reacting, alkylating (alkylation), isomerizing (isomerization), disproportionating (disproportionation), and the like and combinations thereof.

Generally, the reactants comprising ethylene and a hydrocarbon selected from the group consisting of paraffins, isoparaffins, and the like and combinations thereof, are initially present in a hydrocarbon-containing fluid. However, an additional embodiment of a process of the present invention includes separate feed streams comprising a feed stream comprising a hydrocarbon selected from the group consisting of paraffins, isoparaffins, and the like and combinations thereof, such as a fuel gas rich in paraffins, and a separate feed stream comprising ethylene that can be fed separately into a reactor to provide mixing in the presence of a catalyst composition of the present invention.

Examples of suitable hydrocarbon-containing fluids include, but are not limited to, fuel gas, gasolines from catalytic oil cracking (e.g., FCC and hydrocracking) processes, pyrolysis gasolines from thermal hydrocarbon- (e.g., ethane, propane, and naphtha) cracking processes, naphthas, gas oils, reformates, straight-run gasoline, and the like and combinations thereof.

A hydrogen halide component of a catalyst composition of the present invention can be any hydrogen halide component that can be utilized to provide a catalyst composition that can be utilized in a process of the present invention. A hydrogen halide component of a catalyst composition or catalyst mixture of the present invention can be selected from the group of compounds consisting of hydrogen fluoride (HF), hydrogen chloride (HCl), hydrogen bromide (HBr), and the like and combinations thereof. The preferred hydrogen halide component is hydrogen fluoride that can be utilized in the catalyst composition preferably in anhydrous form, but can include impurities such as water as long as the amount of such water does not interfere with conducting a process of the present invention. Preferably, water should be minimized in the hydrogen halide component because it will tend to diminish the effect of, and may destroy, a metal halide component of a catalyst composition of the present invention. If water is present, the amount of water present in the hydrogen halide component is less than about 10 weight percent. Most preferably, the amount of water present in the hydrogen halide component is less than about 5 weight percent. When referring herein to a hydrogen halide component, preferably a hydrogen fluoride component, of a catalyst composition of the present invention, it should be understood that these terms mean either the hydrogen halide component as an anhydrous mixture or a mixture that includes water. The references herein to weight percent water contained in the hydrogen halide component means the ratio of the weight of water to the sum weight of the water and hydrogen halide multiplied by a factor of 100 to place the weight ratio in terms of percent.

A sulfone component of a catalyst composition of the present invention can be any sulfone that can be utilized to provide a catalyst composition that can be utilized in a process of the present invention. The sulfones suitable for use in a catalyst composition of the present invention include the sulfones of the general formula:

R—SO₂—R′

wherein R and R′ are monovalent hydrocarbon alkyl or aryl substituents, each containing from 1 to 8 carbon atoms. Examples of such substituents include dimethylsulfone, dipropylsulfone, diphenylsulfone, ethylmethylsulfone, and the alicyclic sulfones wherein the SO₂ group is bonded to a hydrocarbon ring. In such a case, R and R′ are forming together a branched or unbranched hydrocarbon divalent moiety preferably containing from three to twelve carbon atoms. Among the latter, tetramethylenesulfone or sulfolane, 3-methylsulfolane and 2,4-dimethylsulfolane are more particularly suitable since they offer the advantage of being liquid at conversion conditions of concern herein. These sulfones may also have substituents, particularly one or more halogen atoms, such as for example, chloromethylethylsulfone. These sulfones may advantageously be used in the form of mixtures. Preferably, the sulfone component is sulfolane, preferably in anhydrous form.

A metal halide component of a catalyst composition of the present invention can be any metal halide component that can be utilized to provide a catalyst composition that can be utilized in a process of the present invention. Examples of a suitable metal of the metal halide component include, but are not limited to, metals of Groups III, IV and V of the Periodic Table of Elements. Preferably, a metal of the metal halide component of a catalyst composition of the present invention includes, but is not limited to, B, Al, Ga, In, Sn, Ti, Zr, P, As, Sb, Bi, V, Nb, Ta, and the like and combinations thereof. Preferably, such metal is Ti. A halide of a metal halide component of a catalyst composition of the present invention includes, but is not limited to, fluoride, bromide, chloride, and the like and combinations thereof. Examples of a suitable metal halide component of a catalyst composition of the present invention includes, but is not limited to, SbF₅, TaF₅, PF₅, NbF₅, BF₃, SnF₄, TiF₄, AlC1₃, SnCl₄, AlBr₃, and the like and combinations thereof. Preferably, a metal halide component of a catalyst composition of the present invention is TiF₄

Generally, the weight percents of a hydrogen halide component, a sulfone component, and a metal halide component of a catalyst composition of the present invention can be any weight percents that provide for a catalyst composition that can be utilized in the contacting of at least one feed hydrocarbon selected from the group consisting of paraffins, isoparaffins, and the like and combinations thereof and ethylene according to a process of the present invention. Generally, a weight percent of hydrogen halide component, based on the total weight of the catalyst composition, is in a range of from about 50 weight percent to about 90 weight percent, preferably in a range from about 60 weight percent to about 80 weight percent, and more preferably, in a range from about 65 weight percent to about 75 weight percent.

A weight percent of a sulfone component, based on the total weight of the catalyst composition, is generally in the range from about 10 weight percent to about 35 weight percent, preferably in the range of from about 20 weight percent to about 30 weight percent, and more preferably in the range of from about 20 weight percent to about 25 weight percent.

A weight percent of a metal halide component, based on the total weight of the catalyst composition, is generally in the range of from about 0.01 weight percent to about 20 weight percent, preferably in the range of from about 1 weight percent to about 15 weight percent, and more preferably in the range of from about 5 weight percent to about 10 weight percent.

Generally, a catalyst composition of the present invention comprises at least about 50 weight percent and no more than about 90 weight percent hydrogen halide component based on the total weight of the catalyst composition, at least about 10 weight percent and no more than about 35 weight percent sulfone component based on the total weight of the catalyst composition, and at least about 0.01 weight percent and no more than about 20 weight percent metal halide component based on the total weight of the catalyst composition. A preferred catalyst composition of the present invention comprises at least about 60 weight percent and no more than about 80 weight percent hydrogen halide component based on the total weight of the catalyst composition, at least about 20 weight percent and no more than about 30 weight percent sulfone component based on the total weight of the catalyst composition, and at least about 1 weight percent and no more than about 15 weight percent metal halide component based on the total weight of the catalyst composition. A more preferred catalyst composition of the present invention comprises at least about 65 weight percent and no more than about 75 weight percent hydrogen halide component based on the total weight of the catalyst composition, at least about 20 weight percent and no more than about 25 weight percent sulfone component based on the total weight of the catalyst composition, and at least about 5 weight percent and no more than about 10 weight percent metal halide component based on the total weight of the catalyst composition. An even more preferred catalyst composition of the present invention comprises about 70 weight percent hydrogen halide component, about 23 weight percent sulfone component, and about 7 weight percent metal halide component based on the total weight of the catalyst composition.

A catalyst composition of the present invention can be prepared by contacting a hydrogen halide component, a sulfone component, and a metal halide component in any suitable manner and in suitable order as long as a catalyst composition of the present invention is provided that can be utilized in a process of the present invention. Preferably, a catalyst composition of the present invention is prepared by contacting a desired amount of a sulfone component, preferably anhydrous sulfolane, with a desired amount of a metal halide component, preferably TiF₄. The combination of sulfone component/metal halide component is then contacted with a desired amount of a hydrogen halide component, preferably anhydrous hydrofluoric acid. The catalyst components are mixed and then utilized in a process of the present invention.

Generally, a process of the present invention is conducted under conversion conditions in a conversion zone wherein is contained a catalyst composition of the present invention under conversion conditions that provide for contacting, preferably converting, a hydrocarbon selected from the group consisting of paraffins, isoparaffins, and the like and combinations thereof having from three to seven carbon atoms per molecule, preferably a normal paraffin, and ethylene according to a process of the present invention to provide for an isoparaffin having from about four to about nine carbon atoms per molecule. Conversion conditions include any temperature suitable for conducting a process of the present invention. Generally, a temperature of the present invention is generally in the range of from about 0° F. to about 250° F., preferably in the range from about 50° F. to about 225° F., and more preferably in the range of from about 60° F. to about 200° F.

A reaction pressure of a process of the present invention can be any pressure sufficient to provide for a process of the present invention and is generally sufficient to maintain the reactants and products substantially in the liquid phase. The conversion pressures will generally be in the range of from about 40 pounds gauge pressure per square inch (psig) to about 1000 psig, preferably in the range of from about 100 psig to about 750 psig, and more preferably in the range of from about 200 psig to about 500 psig. With all reactants in the liquid phase, increased pressure has no significant effect upon the conversion(s) of the present invention. Ethylene may be initially gaseous and can be compressed and mixed to achieve solubility.

Contact times for the hydrocarbon conversion(s) of a process of the present invention in a conversion zone in the presence of a catalyst composition of the present invention can be any time period that suitably provides for a conversion process of the present invention. Generally, such contact time should be sufficient to provide for essentially complete conversion of ethylene in the reaction zone. Preferably, the contact time is in the range from about from about 0.05 minute to about 2 hours, more preferably in the range of from about 0.05 minute to about 60 minutes.

A process of the present invention can be carried out either as a batch or continuous type of operation, although it is preferred for economic reasons to carry out the process continuously. It has been generally established that in alkylation processes, the more intimate the contact between the hydrocarbon-containing fluid, i.e., feedstock, and the catalyst, the better the quality of alkylate product obtained. With this in mind, a process of the present invention, when operated as a batch operation, is characterized by the use of vigorous mechanical stirring or shaking of the reactants and catalyst composition.

The reaction zone design is not critical, except that sufficient dispersion of the hydrocarbon into the catalyst composition should be achieved under well-mixed conditions. A preferred reactor design is a continuously stirred tank reactor (CSTR) with stirring at about 500 revolutions per minute (rpm).

An example process of the present invention can be conducted by routing a hydrocarbon-containing fluid, such as a fuel gas rich in ethylene and propane, to a reactor containing a catalyst composition of the present invention. After a sufficient time to complete a desired conversion, the reactor contents can then be separated and the upper hydrocarbon layer can be sent back to a traditional alkylation unit settler. The catalyst composition is preferably recycled separately. Regeneration can be accomplished by any method known in the art, for example, by stripping the hydrogen halide component preferably hydrofluoric acid, under anhydrous conditions and sending the stripped metal halide component/sulfone, preferably stripped TiF₄/sulfolane mixture, to a regenerator operating at a temperature in the range of from about 200° F. to about 600° F. and a pressure of about 1000 psig with hydrogen. Additional conversion can be conducted, separately and/or simultaneously, including, but not limited to, alkylation, isomerization, disproportionation, and the like and combinations thereof.

A weight ratio of total hydrocarbon to ethylene can be any weight ratio that suitably provides for a process of the present invention. Generally, a weight ratio of total hydrocarbon to ethylene is at least about 1:1 and no more than about 30:1, preferably at least about 2:1 and no more than about 25:1, and more preferably at least about 2:1 and no more than about 20:1.

Generally, a process of the present invention provides for a conversion of ethylene of at least about 50 weight percent, preferably at least about 80 weight percent, more preferably at least about 90 weight percent, and more preferably at least about 95 weight percent based on the total weight of the ethylene initially present in a process of the present invention.

Generally, a weight ratio of catalyst composition to total hydrocarbon and ethylene is any weight ratio that suitably provides for a process of the present invention. Generally, a weight ratio of catalyst composition to total hydrocarbon and ethylene initially present in a process of the present invention is at least about 0.5:1 and no more than about 20:1, preferably at least about 1:1 and no more than about 15:1, and more preferably at least about 1:1 and no more than about 10:1.

Generally, a weight ratio of hydrogen halide component to total hydrocarbon is at least about 0.01:1 and no more than about 10:1, preferably at least about 0.5:1 and no more than about 4:1. Higher ratios are expected to lead to higher conversions at otherwise equivalent conditions.

An example process of the present invention comprises contacting ethylene and at least one feed hydrocarbon comprising normal butane in the presence of a catalyst composition of the present invention under conversion conditions to provide at least one product hydrocarbon isomer comprising an isobutane and additional isoparaffins containing from about five to about nine carbon atoms per molecule such as isopentane, 2,2-dimethylbutane, 2,3-dimethylbutane, 2-methylpentane, 3-methylpentane, 2,2-dimethylpentane, 2,4-dimethylpentane, 2-methylhexane, 3-methylhexane, and 2,2-dimethylhexane. The isobutane can be provided by the isomerization of normal butane to isobutane and/or through isopentane disproportionation, such as two isopentanes reacting to provide an isobutane and an isohexane. Such disproportionation reaction usually produces high levels of 2-methylpentanes and 3-methylpentanes in the isohexane fraction.

Another example process of the present invention comprises contacting ethylene and at least one feed hydrocarbon comprising isobutane in the presence of a catalyst composition of the present invention under conversion conditions to provide at least one product hydrocarbon isomer comprising an isopentane and additional isoparaffins containing from about five to about nine carbon atoms per molecule such as 2,2-dimethylbutane, 2,3-dimethylbutane, 2-methylpentane, 3-methylpentane, 2,2,4-trimethylpentane, 2,5-dimethylhexane, and 2,4-dimethylhexane. The isoparaffin can be provided by disproportionation, such as two isopentanes reacting to provide an isobutane and an isohexane. Such disproportionation reaction usually produces high levels of 2-methylpentanes and 3-methylpentanes in the isohexane fraction.

Another example process of the present invention comprises contacting ethylene and at least one feed hydrocarbon comprising propane in the presence of a catalyst composition of the present invention under conversion conditions to provide at least one product hydrocarbon isomer comprising an isobutane and additional isoparaffins containing from about five to about nine carbon atoms per molecule such as isopentane, 2,2-dimethylbutane, 2-methylpentane, and 2-methylhexane.

Another example process of the present invention comprises contacting ethylene and at least one feed hydrocarbon comprising a hydrocarbon containing six or more carbon atoms per molecule, preferably normal heptane, in the presence of a catalyst composition of the present invention under conversion conditions to provide at least one product hydrocarbon isomer comprising an isobutane and additional isoparaffins containing from about five to about nine carbon atoms per molecule such as 2-methylhexane, 3-methylhexane, and 2,2-dimethylhexane.

Another example process of the present invention comprises contacting ethylene and at least one feed hydrocarbon comprising isopentane in the presence of a catalyst composition of the present invention under conversion conditions to provide at least one product hydrocarbon isomer comprising an isobutane and additional isoparaffins containing from about five to about nine carbon atoms per molecule such as 2,3-dimethylbutane, 2-methylpentane, 3-methylpentane, 2,4-dimethylpentane, 2-methylhexane, 2,3-dimethylpentane, and 3-methylhexane.

Another example process of the present invention comprises contacting ethylene and at least one feed hydrocarbon comprising isobutane and at least one feed hydrocarbon comprising normal butane in the presence of a catalyst composition of the present invention under conversion conditions to provide at least one product hydrocarbon isomer comprising an isopentane and additional isoparaffins containing from about five to about nine carbon atoms per molecule such as 2,3-dimethylbutane, 2-methylpentane, 3-methylpentane, 2,5-dimethylhexane, and 2,4-dimethylhexane.

Another example process of the present invention comprises contacting ethylene and at least one feed hydrocarbon comprising isobutane and at least one feed hydrocarbon comprising normal butane and at least one feed hydrocarbon comprising isopentane in the presence of a catalyst composition of the present invention under conversion conditions to provide at least one product hydrocarbon isomer comprising an isobutane and additional isoparaffins containing from about five to about nine carbon atoms per molecule such as 2,3-dimethylbutane, 2-methylpentane, 3-methylpentane, 2,4-dimethylpentane, 2-methylhexane, 2,3-dimethylpentane, and 3-methylhexane.

Another example process of the present invention comprises contacting ethylene and at least one feed hydrocarbon comprising normal pentane in a hydrocarbon-containing fluid in the presence of a catalyst composition of the present invention under conversion conditions to provide at least one product hydrocarbon isomer comprising an isobutane and additional isoparaffins containing from about five to about nine carbon atoms per molecule such as isopentane, 2,3-dimethylbutane, 2-methylpentane, 3-methylpentane, 2,4-dimethylpentane, 2-methylhexane, and 3-methylhexane.

A catalyst composition of the present invention may be added by injection directly into a conversion zone or may be mixed with a hydrocarbon-containing fluid containing ethylene and a hydrocarbon selected from the group consisting of paraffins, isoparaffins, and the like and combinations thereof, or may be mixed with fresh and/or circulating catalyst composition, or with a stream of mixed hydrocarbon-containing fluid and catalyst composition. Downstream from the conversion zone, the catalyst composition can be preferably separated from the product stream, mixed with fresh and/or circulating catalyst composition, and recycled to the conversion zone. The particular separation technique selected depends upon the characteristics of the catalyst composition and the desired reaction products. Selection of such separation techniques is within the skill in the art.

The following examples are presented to further illustrate the present invention and are not to be construed as unduly limiting the scope of the present invention. In the following Examples and Tables the following abbreviations are used: Rxn is reaction; C2=is ethylene; C2 is ethane; C2F is fluoroethane; C3 is propane; iC4 is isobutane; nC4 is normal butane; C4F is fluorobutane; UnkC1-C4 is unidentified hydrocarbons containing from one to four carbon atoms per molecule; iC5 is isopentane; nC5 is normal pentane; C6+ is total hydrocarbons containing six or more carbon atoms per molecule; C5+ is total hydrocarbons containing five or more carbon atoms per molecule; 22DMC4 is 2,2-dimethylbutane; 23DMC4 is 2,3-dimethylbutane; 2MC5 is 2-methylpentane; 3MC5 is 3-methylpentane; nC6 is normal hexane; 22DMC5 is 2,2-dimethylpentane; 24DMC5 is 2,4-dimethylpentane; 223TMC4 is 2,2,3-trimethylbutane; 33DMC5 is 3,3-dimethylpentane; 2MC6 is 2-methylhexane; 23DMC5 is 2,3-dimethylpentane; 3MC6 is 3-methylhexane; 3EtC5 is 3-ethylpentane; 224TMC5 is 2,2,4-trimethylpentane; nC7 is normal heptane; 22DMC6 is 2,2-dimethylhexane; 25DMC6 is 2,5-dimethylhexane; 24DMC6 is 2,4-dimethylhexane; 33DMC6 is 3,3-dimethylhexane; 234TMC5 is 2,3,4-trimethylpentane; 233TMC5 is 2,3,3-trimethylpentane; 23DMC6 is 2,3-dimethylhexane; 2M3EtC5 is 2-methyl-3-ethylpentane; 2MC7 is 2-methylheptane; 4MC7 is 4-methylheptane; 34DMC6 is 3,4-dimethylhexane; 3MC7 is 3-methylheptane; 225TMC6 is 2,2,5-trimethylhexane; Residue is all material boiling higher than 2,2,5-trimethylhexane; Unk C5-C8 is unidentified hydrocarbons containing five to eight carbon atoms per molecule; C5+ RON is the Research Octane Number of total hydrocarbons containing five or more carbon atoms per molecule (as estimated from gas chromatography); C6+ RON is the Research Octane Number of total hydrocarbons containing six or more carbon atoms per molecule (as estimated from gas chromatography). Also, regarding the catalyst, HF is hydrofluoric acid, TiF4 is titanium tetrafluoride, and HF/S w/w is the weight ratio of hydrofluoric acid to sulfolane. All numbers in the Tables are weight percent unless otherwise indicated.

### EXAMPLE 1

Example 1 illustrates a process of the present invention comprising contacting normal butane and ethylene in a hydrocarbon-containing fluid in the presence of a catalyst composition under conversion conditions to provide isobutane and additional reaction products comprising isoparaffins having from about five to about nine carbon atoms per molecule wherein the catalyst composition comprises hydrofluoric acid, sulfolane, and TiF₄.

A catalyst composition was prepared as follows. A clean, dry 300 cubic centimeters (cc) Monel cylinder was charged with the desired amount of anhydrous sulfolane followed by the addition of the desired amount of TiF₄. A valve was then attached to the cylinder and the desired amount of hydrofluoric acid was added from a supply of anhydrous hydrofluoric acid. The cylinder was removed from the hydrofluoric acid source, shaken, and then charged to a batch reactor system.

The batch reactor system consisted of a Monel autoclave (300 mL volume) equipped with a mechanical stirrer, a heater, a thermocouple attached to a temperature controller, a pressure gauge, various valves, and two Monel sight glasses used for hydrocarbon-containing fluid feed introduction and product settling. After charging the catalyst composition, the temperature controller was set to achieve the desired temperature. Stirring was initiated at 500 revolutions per minute (rpm). The hydrocarbon-containing fluid feed was blended gravimetrically to a 500 mL stainless steel cylinder. The higher boiling component(s) was added first, followed by attachment of the cylinder to a supply of ethylene. The desired amount of ethylene was then added and the cylinder was removed and weighed. After analysis by gas chromatography, the feed cylinder was attached to a 150 mL sight glass used for the hydrocarbon-containing fluid feed addition. The hydrocarbon-containing fluid feed was added to the Monel reactor via pressure differential over a period of about 30 to 60 seconds. The reaction was allowed to proceed for the desired length of time at the desired temperature. All of the conversion reactions were conducted at a pressure of about 400 psig.

At the desired time, the stirring was stopped and the reactor contents were transferred to a second Monel sight glass used as a settler. The acid components settled to the bottom of the gauge and were removed into a Monel cylinder for further use, analysis, or destruction. The hydrocarbon phase was then collected into a stainless steel cylinder containing 100 mL of 1.5N potassium hydroxide solution to neutralize any acid species. The water layer was removed and the hydrocarbon layer was analyzed by gas chromatography.

Table 1 discloses the amounts and components of the catalyst composition, the components of the hydrocarbon-containing fluid feed, and a summary product composition analysis. Table 2 discloses a detailed product composition analysis. The test data in Tables 1 and 2 clearly show that the inventive process converted over 90% of the ethylene. Further, the data demonstrate that about 70% of the normal butane was converted. The data also demonstrate that a process of the present invention is effective in contacting normal butane and ethylene in the presence of a catalyst composition under conversion conditions to provide at least one product hydrocarbon isomer comprising isobutane and additional isoparaffins containing from about five to about nine carbon atoms per molecule such as isopentane, 2,2-dimethylbutane, 2,3-dimethylbutane, 2-methylpentane, 3-methylpentane, 2,2-dimethylpentane, 2,4-dimethylpentane, 2-methylhexane, 3-methylhexane, and 2,2-dimethylhexane. Such data is also significant when considering the moderate conversion conditions.

TABLE I

| Component | Wt % |
|---|---|
| Feed | Rxn 1 |
| C= | 7.399 |
| C2F | 0 |
| C3 | 0.002 |
| iC4 | 0.912 |
| nC4 | 90.888 |
| C4F | 0 |
| UnkC1-C4 | 0.734 |
| iC5 | 0.060 |
| nC5 | 0.001 |
| C6+ | 0.004 |
| Total | 100.000 |
| Feed wt, g | 48.9 |
| Catalyst: | |
| HF, g | 68.88 |
| TiF4, g | 22.96 |
| Sulfolane, g | 7.64 |
| Total | 99.48 |
| Mol % TiF4 | 5.0 |
| HF/S w/w | 9.02 |
| Temp, ° F. | 141.0 |
| Time, min | 30.0 |
| Settler Effluent Product (Summary) | |
| C= | 0.897 |
| C2F | 0.005 |
| C3 | 1.940 |
| iC4 | 39.426 |
| nC4 | 26.780 |
| C4F | 0 |
| Unk C1-C5 | 0.747 |
| C5+ | 30.205 |
| Total | 100.000 |

TABLE 2
Settler Effluent Product (Detailed)

| | Rxn 1 |
|---|---|
| Component | (wt. %) |
| C2= | 0.897 |
| C2F | 0.005 |
| C3 | 1.940 |
| iC4 | 39.426 |
| nC4 | 26.780 |
| UnkC1-C4 | 0.747 |
| iC5 | 14.903 |
| nC5 | 3.114 |
| 22DMC4 | 3.308 |
| 23DMC4 | 0.886 |
| 2MC5 | 2.324 |
| 3MC5 | 1.133 |
| nC6 | 0.629 |
| 22DMC5 | 0.232 |
| 24DMC5 | 0.251 |
| 223TMC4 | 0.111 |
| 33DMC5 | 0.187 |
| 2MC6 | 0.442 |
| 23DMC5 | 0.166 |
| 3MC6 | 0.334 |
| 3EtC5 | 0.015 |
| 224TMC5 | 0.040 |
| nC7 | 0.123 |
| 22DMC6 | 0.231 |
| 25DMC6 | 0.186 |
| 24DMC6 | 0.185 |
| 33DMC6 | 0.079 |
| 234TMC5 | 0.008 |
| 233TMC5 | 0.013 |
| 23DMC6 | 0.059 |
| 2M3EtC5 | 0.004 |
| 2MC7 | 0.187 |
| 4MC7 | 0.054 |
| 34DMC6 | 0.020 |
| 3MC7 | 0.165 |
| 225TMC6 | 0.091 |
| Residue | 0.696 |
| Unk C5-C8 | 0.031 |
| Total | 100.000 |
| C5+ RON | 83.6 |
| C6+ RON | |

### EXAMPLE 2

Example 2 illustrates a process of the present invention comprising contacting isobutane and ethylene in a hydrocarbon-containing fluid in the presence of a catalyst composition under conversion conditions to provide an isopentane and additional reaction products comprising isoparaffins having from about five to about nine carbon atoms per molecule wherein the catalyst composition comprises hydrofluoric acid, sulfolane, and TiF₄.

The catalyst composition preparation and reactor system described herein in Example 1 was utilized. Table 3 discloses the amounts and components of the catalyst composition, the components of the hydrocarbon-containing fluid feed, and a summary product composition analysis. Table 4 discloses a detailed product composition analysis. The test data in Tables 3 and 4 clearly show that the inventive process converted over 90% of the ethylene. The data clearly demonstrate that a process of the present invention is effective in contacting isobutane and ethylene in the presence of a catalyst composition of the present invention under conversion conditions to provide at least one product hydrocarbon isomer comprising isopentane and additional isoparaffins containing from about five to about nine carbon atoms per molecule such as 2,2-dimethylbutane, 2,3-dimethylbutane, 2-methylpentane, 3-methylpentane, 2,2,4-trimethylpentane, 2,5-dimethylhexane, and 2,4-dimethylhexane.

TABLE 3

| Component | Wt % |
|---|---|
| Feed | Rxn 2 |
| C2= | 8.033 |
| C2F | 0 |
| C3 | 0.333 |
| iC4 | 89.210 |
| nC4 | 2.407 |
| C4F | 0 |
| UnkC1-C4 | 0.015 |
| iC5 | 0.002 |
| nC5 | 0.000 |
| C6+ | 0.000 |
| Total | 100.000 |
| Feed wt, g | 48.4 |
| Catalyst: | |
| HF, g | 68.88 |
| TiF4, g | 22.96 |
| Sulfolane, g | 7.64 |
| Total | 99.48 |
| Mol % TiF4 | 5.0 |
| HF/S w/w | 9.02 |
| Temp, ° F. | 104.5 |
| Time, min | 30.0 |
| Settler Effluent Product (Summary) | |
| C2= | 0.189 |
| C2F | 0.921 |
| C3 | 0.562 |
| iC4 | 86.467 |
| nC4 | 4.222 |
| C4F | 0.050 |
| Unk C1-C5 | 0.005 |
| C5+ | 7.584 |
| Total | 100.000 |

TABLE 4
Settler Effluent Product (Detailed)

| | Rxn 2 |
|---|---|
| Component | (wt. %) |
| C2= | 0.189 |
| C2F | 0.921 |
| C3 | 0.562 |
| iC4 | 86.467 |
| nC4 | 4.222 |
| Unk C1-C4 | 0.054 |
| iC5 | 1.360 |
| nC5 | 0.119 |
| 22DMC4 | 0.410 |
| 23DMC4 | 2.006 |
| 2MC5 | 1.167 |
| 3MC5 | 0.527 |
| nC6 | 0.027 |
| 22DMC5 | 0.021 |
| 24DMC5 | 0.044 |
| 223TMC4 | 0.006 |
| 33DMC5 | 0.007 |
| 2MC6 | 0.031 |
| 23DMC5 | 0.055 |
| 3MC6 | 0.022 |
| 3EtC5 | 0.000 |
| 224TMC5 | 0.378 |
| nC7 | 0.002 |
| 22DMC6 | 0.014 |
| 25DMC6 | 0.354 |
| 24DMC6 | 0.403 |
| 33DMC6 | 0.003 |
| 234TMC5 | 0.057 |
| 233TMC5 | 0.105 |
| 23DMC6 | 0.099 |
| 2M3EtC5 | 0.005 |
| 2MC7 | 0.095 |
| 4MC7 | 0.025 |
| 34DMC6 | 0.030 |
| 3MC7 | 0.075 |
| 225TMC6 | 0.040 |
| Residue | 0.095 |
| UnkC5-C8 | 0.004 |
| Total | 100.001 |
| C5+ RON | 86.5 |
| C6+ RON | |

### EXAMPLE 3

Example 3 illustrates that a process of the present invention is not as effective in converting a hydrocarbon-containing fluid comprising entirely ethylene to isoparaffins under conversion conditions similar to the conversion conditions utilized when contacting a hydrocarbon-containing fluid comprising paraffins containing three or more carbon atoms per molecule and ethylene according to a process of the present invention. The catalyst composition preparation and reactor system described herein in Example 1 was utilized. Table 5 discloses the amounts and components of the catalyst composition, the components of the hydrocarbon-containing fluid feed, and a summary product composition analysis. Table 6 discloses a detailed product composition analysis. The test data in Tables 5 and 6 clearly show that the inventive process was not as effective in converting a hydrocarbon-containing fluid comprising entirely ethylene to isoparaffins under conversion conditions similar to the conversion conditions utilized when contacting a hydrocarbon-containing fluid comprising paraffins containing three or more carbon atoms per molecule and ethylene according to a process of the present invention.

TABLE 5

| Component | Wt % |
|---|---|
| C2= | 100 |
| C2F | |
| C3 | |
| iC4 | |
| nC4 | |
| C4F | |
| UnkC1-C4 | |
| iC5 | |
| nC5 | |
| C6+ | |
| Total | 100.000 |
| Feed wt, g | 7.5 |
| Catalyst: | |
| HF, g | 69.74 |
| TiF4, g | 22.95 |
| Sulfolane, g | 7.63 |
| Total | 100.32 |
| Mol % TiF4 | 5.0 |
| HF/S w/w | 9.14 |
| Temp, ° F. | 151.0 |
| Time, min | 60.0 |
| Settler Effluent Product (Summary) | |
| C2= | NO |
| C2F | RXN |
| C3 | |
| iC4 | |
| nC4 | |
| C4F | |
| Unk C1-C5 | |
| C5+ | |
| Total | |

TABLE 6
Settler Effluent Product (Detailed)

| | Rxn 3 |
|---|---|
| Component | (wt. %) |
| C2= | NO |
| C2F | RXN |
| C3 | |
| iC4 | |
| nC4 | |
| Unk C1-C4 | |
| iC5 | |
| nC5 | |
| 22DMC4 | |
| 23DMC4 | |
| 2MC5 | |
| 3MC5 | |
| nC6 | |
| 22DMC5 | |
| 24DMC5 | |
| 223TMC4 | |
| 33DMC5 | |
| 2MC6 | |
| 23DMC5 | |
| 3MC6 | |
| 3EtC5 | |
| 224TMC5 | |
| nC7 | |
| 22DMC6 | |
| 25DMC6 | |
| 24DMC6 | |
| 33DMC6 | |
| 234TMC5 | |
| 233TMC5 | |
| 23DMC6 | |
| 2M3EtC5 | |
| 2MC7 | |
| 4MC7 | |
| 34DMC6 | |
| 3MC7 | |
| 225TMC6 | |
| Residue | |
| Unk C5-C8 | |
| Total | 0.000 |
| C5+ RON | |
| C6+ RON | |

### EXAMPLE 4

Example 4 illustrates a process of the present invention comprising contacting propane and ethylene in a hydrocarbon-containing fluid in the presence of a catalyst composition under conversion conditions to provide isobutane and additional reaction products comprising isoparaffins having from about five to about nine carbon atoms per molecule wherein the catalyst composition comprises hydrofluoric acid, sulfolane, and TiF₄.

The catalyst composition preparation and reactor system described herein in Example 1 was utilized. Table 7 discloses the amounts and components of the catalyst composition, the components of the hydrocarbon-containing fluid feed, and a summary product composition analysis. Table 8 discloses a detailed product composition analysis. The test data in Tables 7 and 8 clearly show that the inventive process converted over 90% of the ethylene. The data clearly demonstrate that a process of the present invention is effective in contacting propane and ethylene in the presence of a catalyst composition of the present invention under conversion conditions to provide at least one product hydrocarbon isomer comprising isobutane and additional isoparaffins containing from about five to about nine carbon atoms per molecule such as isopentane, 2-methylpentane, and 2-methylhexane.

TABLE 7

| Component | Wt % |
|---|---|
| Feed | Rxn 4 |
| C2= | 8.649 |
| C2F | 0 |
| C3 | 91.010 |
| iC4 | 0.296 |
| nC4 | 0.009 |
| C4F | 0 |
| UnkC1-C4 | 0.035 |
| iC5 | 0.000 |
| nC5 | 0.000 |
| C6+ | 0.000 |
| Total | 100.000 |
| Feed wt, g | 46.4 |
| Catalyst: | |
| HF, g | 69.74 |
| TiF4, g | 22.95 |
| Sulfolane, g | 7.63 |
| Total | 100.32 |
| Mol % TiF4 | 5.0 |
| HF/S w/w | 9.14 |
| Temp, ° F. | 139.9 |
| Time, min | 30.0 |
| Settler Effluent Product (Summary) | |
| C2= | 0.463 |
| C2F | 0.365 |
| C3 | 85.857 |
| iC4 | 3.656 |
| nC4 | 0.781 |
| C4F | 0.002 |
| Unk C1-C5 | 0.023 |
| C5+ | 8.853 |
| Total | 100.000 |

TABLE 8
Settler Effluent Product (Detailed)

| | Rxn 4 |
|---|---|
| Component | (wt. %) |
| C2= | 0.463 |
| C2F | 0.365 |
| C3 | 85.857 |
| iC4 | 3.656 |
| nC4 | 0.781 |
| Unk C1-C4 | 0.025 |
| iC5 | 2.828 |
| nC5 | 0.446 |
| 22DMC4 | 0.448 |
| 23DMC4 | 0.397 |
| 2MC5 | 1.009 |
| 3MC5 | 0.480 |
| nC6 | 0.203 |
| 22DMC5 | 0.079 |
| 24DMC5 | 0.340 |
| 223TMC4 | 0.140 |
| 33DMC5 | 0.077 |
| 2MC6 | 0.527 |
| 23DMC5 | 0.216 |
| 3MC6 | 0.392 |
| 3EtC5 | 0.018 |
| 224TMC5 | 0.005 |
| nC7 | 0.102 |
| 22DMC6 | 0.054 |
| 25DMC6 | 0.107 |
| 24DMC6 | 0.097 |
| 33DMC6 | 0.012 |
| 234TMC5 | 0.001 |
| 233TMC5 | 0.002 |
| 23DMC6 | 0.032 |
| 2M3EtC5 | 0.002 |
| 2MC7 | 0.114 |
| 4MC7 | 0.032 |
| 34DMC6 | 0.011 |
| 3MC7 | 0.097 |
| 225TMC6 | 0.029 |
| Residue | 0.506 |
| Unk C5-C8 | 0.050 |
| Total | 100.000 |
| C5+ RON | 78.5 |
| C6+ RON | |

### EXAMPLE 5

The following example illustrates that a process of the present invention is not as effective in converting a hydrocarbon-containing fluid comprising entirely ethane and ethylene to isoparaffins under conversion conditions similar to the conversion conditions utilized when contacting a hydrocarbon-containing fluid comprising paraffins containing three or more carbon atoms per molecule and ethylene according to a process of the present invention. The catalyst composition preparation and reactor system described herein in Example 1 was utilized. Table 9 discloses the amounts and components of the catalyst composition, the components of the hydrocarbon-containing fluid feed, and a summary product composition analysis. Table 10 discloses a detailed product composition analysis. The test data in Tables 9 and 10 clearly demonstrate that the inventive process was not as effective in converting a hydrocarbon-containing fluid comprising entirely ethane and ethylene to isoparaffins under conversion conditions similar to the conversion conditions utilized when contacting a hydrocarbon-containing fluid comprising paraffins containing three or more carbon atoms per molecule and ethylene according to a process of the present invention.

TABLE 9

| Component | Wt % |
|---|---|
| Feed | Rxn 5 |
| C2= | 20 |
| C2F | |
| C3 | |
| iC4 | |
| nC4 | |
| C4F | |
| UnkC1-C4 | 80 (C2) |
| iC5 | |
| nC5 | |
| C6+ | 0 |
| Total | 100 |
| Feed wt, g | 15.0 |
| Catalyst: | |
| HF, g | 69.1 |
| TiF4, g | 22.93 |
| Sulfolane, g | 7.65 |
| Total | 99.68 |
| Mol % TiF4 | 5.0 |
| HF/S w/w | 9.03 |
| Temp, ° F. | 140-180 |
| Time, min | 18+ hrs |
| Settler Effluent Product (Summary) | |
| C2= | NO |
| C2F | RXN |
| C3 | |
| iC4 | |
| nC4 | |
| C4F | |
| Unk C1-C5 | |
| C5+ | |
| Total | |

TABLE 10
Settler Effluent Product (Detailed)

| | Rxn 5 |
|---|---|
| Component | (wt. %) |
| C2= | NO |
| C2F | RXN |
| C3 | |
| iC4 | |
| nC4 | |
| Unk C1-C4 | |
| iC5 | |
| nC5 | |
| 22DMC4 | |
| 23DMC4 | |
| 2MC5 | |
| 3MC5 | |
| nC6 | |
| 22DMC5 | |
| 24DMC5 | |
| 223TMC4 | |
| 33DMC5 | |
| 2MC6 | |
| 23DMC5 | |
| 3MC6 | |
| 3EtC5 | |
| 224TMC5 | |
| nC7 | |
| 22DMC6 | |
| 25DMC6 | |
| 24DMC6 | |
| 33DMC6 | |
| 234TMC5 | |
| 233TMC5 | |
| 23DMC6 | |
| 2M3EtC5 | |
| 2MC7 | |
| 4MC7 | |
| 34DMC6 | |
| 3MC7 | |
| 225TMC6 | |
| Residue | |
| Unk C5-C8 | |
| Total | 0.000 |
| C5+ RON | |
| C6+ RON | |

### EXAMPLE 6

Example 6 illustrates a process of the present invention comprising contacting a hydrocarbon containing six or more carbon atoms per molecule and ethylene in a hydrocarbon-containing fluid in the presence of a catalyst composition under conversion conditions to provide isobutane and additional reaction products comprising isoparaffins having from about five to about nine carbon atoms per molecule wherein the catalyst composition comprises hydrofluoric acid, sulfolane, and TiF₄.

The catalyst composition preparation and reactor system described herein in Example 1 was utilized. Table 11 discloses the amounts and components of the catalyst composition, the components of the hydrocarbon-containing fluid feed, and a summary product composition analysis. Table 12 discloses a detailed product composition analysis. The test data in Tables 11 and 12 clearly show that the inventive process converted over 90% of the ethylene. The data clearly demonstrate that a process of the present invention is effective in contacting hydrocarbons containing six or more carbon atoms per molecule, such as normal heptane, and ethylene in the presence of a catalyst composition of the present invention under conversion conditions to provide at least one product hydrocarbon isomer comprising isobutane and additional isoparaffins containing from about five to about nine carbon atoms per molecule such as 2-methylhexane, 3-methylhexane, and 2,2-dimethylhexane.

TABLE 11

| Component | Wt % |
|---|---|
| Feed | Rxn 6 |
| C2= | 5.647 |
| C2F | 0 |
| C3 | 0 |
| iC4 | 0.002 |
| nC4 | 0 |
| C4F | 0 |
| UnkC1-C4 | 0 |
| iC5 | 0.001 |
| nC5 | 0 |
| C6+ | 94.351 |
| Total | 100.000 |
| Feed wt, g | 48.6 |
| Catalyst: | |
| HF, g | 69.74 |
| TiF4, g | 22.95 |
| Sulfolane, g | 7.63 |
| Total | 100.32 |
| Mol % TiF4 | 5.0 |
| HF/s w/w | 9.14 |
| Temp, ° F. | 102.7 |
| Time, min | 30 |
| Settler Effluent Product (Summary) | |
| C2= | 0.138 |
| C2F | 0.502 |
| C3 | 0.213 |
| iC4 | 0.362 |
| nC4 | 0.110 |
| C4F | 0.000 |
| Unk C1-C5 | 0.000 |
| C5+ | 98.675 |
| Total | 100.000 |

TABLE 12
Settler Effluent Product (Detailed)

| | Rxn 6 |
|---|---|
| Component | (wt. %) |
| C2= | 0.138 |
| C2F | 0.502 |
| C3 | 0.213 |
| iC4 | 0.362 |
| nC4 | 0.110 |
| Unk C1-C4 | 0.000 |
| iC5 | 0.130 |
| nC5 | 0.017 |
| 22DMC4 | 0.051 |
| 23DMC4 | 0.033 |
| 2MC5 | 0.049 |
| 3MC5 | 0.021 |
| nC6 | 0.004 |
| 22DMC5 | 0.005 |
| 24DMC5 | 0.193 |
| 223TMC4 | 0.005 |
| 33DMC5 | 0.016 |
| 2MC6 | 0.661 |
| 23DMC5 | 0.139 |
| 3MC6 | 0.565 |
| 3EtC5 | 0.004 |
| 224TMC5 | 0.024 |
| nC7 | 95.768 |
| 22DMC6 | 0.517 |
| 25DMC6 | 0.035 |
| 24DMC6 | 0.046 |
| 33DMC6 | 0.007 |
| 234TMC5 | 0.000 |
| 233TMC5 | 0.000 |
| 23DMC6 | 0.009 |
| 2M3EtC5 | 0.000 |
| 2MC7 | 0.020 |
| 4MC7 | 0.005 |
| 34DMC6 | 0.002 |
| 3MC7 | 0.015 |
| 225TMC6 | 0.002 |
| Residue | 0.302 |
| Unk C5-C8 | 0.028 |
| Total | 100.000 |
| C5+ RON | |
| C6+ RON | |

### EXAMPLE 7

Example 7 illustrates a process of the present invention comprising contacting isopentane and ethylene in a hydrocarbon-containing fluid in the presence of a catalyst composition under conversion conditions to provide isobutane and additional reaction products comprising isoparaffins having from about five to about nine carbon atoms per molecule wherein the catalyst composition comprises hydrofluoric acid, sulfolane, and TiF₄.

The catalyst composition preparation and reactor system described herein in Example 1 was utilized. Table 13 discloses the amounts and components of the catalyst composition, the components of the hydrocarbon-containing fluid feed, and a summary product composition analysis. Table 14 discloses a detailed product composition analysis. The test data in Tables 13 and 14 clearly show that the inventive process converted over 90% of the ethylene. The data clearly demonstrate that a process of the present invention is effective in contacting isopentane and ethylene in the presence of a catalyst composition of the present invention under conversion conditions to provide at least one product hydrocarbon isomer comprising isobutane and additional isoparaffins containing from about five to about nine carbon atoms per molecule such as 2,3-dimethylbutane, 2-methylpentane, 3-methylpentane, 2,4-dimethylpentane, 2-methylhexane, 2,3-dimethylpentane, and 3-methylhexane.

TABLE 13

| Component | Wt % |
|---|---|
| Feed | Rxn 7 |
| C2= | 6.417 |
| C2F | 0 |
| C3 | 0.002 |
| iC4 | 0.038 |
| nC4 | 0.085 |
| C4F | 0 |
| UnkC1-C4 | 0.209 |
| iC5 | 92.750 |
| nC5 | 0.456 |
| C6+ | 0.043 |
| Total | 100.000 |
| Feed wt, g | 47.4 |
| Catalyst: | |
| HF, g | 69.52 |
| TiF4, g | 22.96 |
| Sulfolane, g | 7.64 |
| Total | 100.12 |
| Mol % TiF4 | 5.0 |
| HF/S w/w | 9.10 |
| Temp, ° F. | 120.7 |
| Time, min | 10 |
| Settler Effluent Product (Summary) | |
| C2= | 0.104 |
| C2F | 0.603 |
| C3 | 0.487 |
| iC4 | 11.224 |
| nC4 | 0.847 |
| C4F | 0.000 |
| Unk C1-C5 | 0.201 |
| C5+ | 86.534 |
| Total | 100.000 |

TABLE 14
Settler Effluent Product (Detailed)

| | Rxn 7 |
|---|---|
| Component | (wt %) |
| C2= | 0.104 |
| C2F | 0.603 |
| C3 | 0.487 |
| iC4 | 11.224 |
| nC4 | 0.847 |
| Unk C1-C4 | 0.201 |
| iC5 | 65.413 |
| nC5 | 1.175 |
| 22DMC4 | 0.237 |
| 23DMC4 | 2.078 |
| 2MC5 | 7.972 |
| 3MC5 | 3.862 |
| nC6 | 0.073 |
| 22DMC5 | 0.109 |
| 24DMC5 | 1.022 |
| 223TMC4 | 0.044 |
| 33DMC5 | 0.014 |
| 2MC6 | 0.960 |
| 23DMC5 | 0.637 |
| 3MC6 | 0.709 |
| 3EtC5 | 0.031 |
| 224TMC5 | 0.007 |
| nC7 | 0.183 |
| 22DMC6 | 0.010 |
| 25DMC6 | 0.093 |
| 24DMC6 | 0.083 |
| 33DMC6 | 0.001 |
| 234TMC5 | 0.001 |
| 233TMC5 | 0.002 |
| 23DMC6 | 0.028 |
| 2M3EtC5 | 0.002 |
| 2MC7 | 0.063 |
| 4MC7 | 0.018 |
| 34DMC6 | 0.008 |
| 3MC7 | 0.052 |
| 225TMC6 | 0.242 |
| Residue | 1.319 |
| Unk C5-C8 | 0.087 |
| Total | 100.000 |
| C5+ RON | 89.2 |
| C6+ RON | 75.1 |

### EXAMPLE 8

Example 8 illustrates the effects of higher temperature and shorter contact time on a process of the present invention comprising contacting isobutane and ethylene in a hydrocarbon-containing fluid in the presence of a catalyst composition of the present invention under conversion conditions to provide at least one product hydrocarbon isomer comprising isopentane and additional isoparaffins containing from about five to about nine carbon atoms per molecule wherein the catalyst composition comprises hydrofluoric acid, sulfolane, and TiF₄.

The catalyst composition preparation and reactor system described herein in Example 1 was utilized. Table 15 discloses the amounts and components of the catalyst composition, the components of the hydrocarbon-containing fluid feed, and a summary product composition analysis. Table 16 discloses a detailed product composition analysis. The test data in Tables 15 and 16 clearly show that the inventive process converted over 90% of the ethylene. The data clearly demonstrate that a process of the present invention is effective in contacting isobutane and ethylene in the presence of a catalyst composition of the present invention under conversion conditions to provide at least one product hydrocarbon isomer comprising isopentane and additional isoparaffins containing from about five to about nine carbon atoms per molecule such as 2,3-dimethylbutane, 2-methylpentane, 3-methylpentane, 2,5-dimethylhexane, and 2,4-dimethylhexane.

TABLE 15

| Component | Wt % |
|---|---|
| Feed | Rxn 8 |
| C2= | 8.785 |
| C2F | 0 |
| C3 | 0.325 |
| iC4 | 88.481 |
| nC4 | 2.390 |
| C4F | 0 |
| UnkC1-C4 | 0.017 |
| iC5 | 0.002 |
| nC5 | 0.000 |
| C6+ | 0.000 |
| Total | 100.000 |
| Feed wt, g | 48.3 |
| Catalyst: | |
| HF, g | 68.93 |
| TiF4, g | 22.91 |
| Sulfolane, g | 7.65 |
| Total | 99.49 |
| Mol % TiF4 | 5.0 |
| HF/S w/w | 9.01 |
| Temp, ° F. | 119.9 |
| Time, min | 10 |
| Settler Effluent Product (Summary) | |
| C2= | 0.329 |
| C2F | 0.670 |
| C3 | 0.402 |
| iC4 | 79.907 |
| nC4 | 4.622 |
| C4F | 0.003 |
| Unk C1-C5 | 0.004 |
| C5+ | 14.063 |
| Total | 100.000 |

TABLE 16
Settler Effluent Product (Detailed)

| | |
|---|---|
| | Rxn 8 |
| Component | (wt %) |

| | |
|---|---|
| C2= | 0.329 |
| C2F | 0.670 |
| C3 | 0.402 |
| iC4 | 79.907 |
| nC4 | 4.622 |
| Unk C1-C4 | 0.004 |
| iC5 | 4.364 |
| nC5 | 0.088 |
| 22DMC4 | 0.514 |
| 23DMC4 | 1.770 |
| 2MC5 | 2.199 |
| 3MC5 | 1.027 |
| nC6 | 0.096 |
| 22DMC5 | 0.003 |
| 24DMC5 | 0.153 |
| 223TMC4 | 0.012 |
| 33DMC5 | 0.005 |
| 2MC6 | 0.179 |
| 23DMC5 | 0.092 |
| 3MC6 | 0.129 |
| 3EtC5 | 0.006 |
| 224TMC5 | 0.311 |
| nC7 | 0.019 |
| 22DMC6 | 0.059 |
| 25DMC6 | 0.626 |
| 24DMC6 | 0.655 |
| 33DMC6 | 0.013 |
| 234TMC5 | 0.049 |
| 233TMC5 | 0.088 |
| 23DMC6 | 0.181 |
| 2M3EtC5 | 0.011 |
| 2MC7 | 0.380 |
| 4MC7 | 0.106 |
| 34DMC6 | 0.058 |
| 3MC7 | 0.313 |
| 225TMC6 | 0.104 |
| Residue | 0.437 |
| Unk C5-C8 | 0.020 |
| Total | 100.000 |
| C5+ RON | 82.4 |
| C6+ RON | 76.1 |

### EXAMPLE 9

Example 9 illustrates the effect of a lower temperature on a process of the present invention comprising contacting isobutane and ethylene in a hydrocarbon-containing fluid in the presence of a catalyst composition of the present invention under conversion conditions to provide at least one product hydrocarbon isomer comprising an isopentane and additional isoparaffins containing from about five to about nine carbon atoms per molecule wherein the catalyst composition comprises hydrofluoric acid, sulfolane, and TiF₄.

The catalyst composition preparation and reactor system described herein in Example 1 was utilized. Table 17 discloses the amounts and components of the catalyst composition, the components of the hydrocarbon-containing fluid feed, and a summary product composition analysis. Table 18 discloses a detailed product composition analysis. The test data in Tables 17 and 18 clearly show that the inventive process converted over 90% of the ethylene. The data clearly demonstrate that a process of the present invention is effective in contacting isobutane in a hydrocarbon-containing fluid and ethylene in the presence of a catalyst composition of the present invention under conversion conditions to provide at least one product hydrocarbon isomer comprising isopentane and additional isoparaffins containing from about five to about nine carbon atoms per molecule such as 2,3-dimethylbutane, 2-methylpentane, 3-methylpentane, 2,5-dimethylhexane, and 2,4-dimethylhexane.

TABLE 17

| Component | Wt % |
|---|---|
| Feed | Rxn 9 |
| C2= | 8.577 |
| C2F | 0 |
| C3 | 0.331 |
| iC4 | 88.683 |
| nC4 | 2.391 |
| C4F | 0 |
| UnkC1-C4 | 0.017 |
| iC5 | 0.001 |
| nC5 | 0.000 |
| C6+ | 0.000 |
| Total | 100.000 |
| Feed wt, g | 47.2 |
| Catalyst: | |
| HF, g | 68.93 |
| TiF4, g | 22.91 |
| Sulfolane, g | 7.65 |
| Total | 99.49 |
| Mol % TiF4 | 5.0 |
| HF/S w/w | 9.01 |
| Temp, ° F. | 97.1 |
| Time, min | 30 |
| Settler Effluent Product (Summary) | |
| C2= | 0.306 |
| C2F | 0.991 |
| C3 | 0.964 |
| iC4 | 85.121 |
| nC4 | 3.920 |
| C4F | 0.001 |
| Unk C1-C5 | 0.006 |
| C5+ | 8.691 |
| Total | 100.000 |

TABLE 18
Settler Effluent Product (Detailed)

| | Rxn 9 |
|---|---|
| Component | (wt %) |
| C2= | 0.306 |
| C2F | 0.991 |
| C3 | 0.964 |
| iC4 | 85.121 |
| nC4 | 3.920 |
| Unk C1-C4 | 0.007 |
| iC5 | 1.565 |
| nC5 | 0.124 |
| 22DMC4 | 0.482 |
| 23DMC4 | 2.409 |
| 2MC5 | 1.359 |
| 3MC5 | 0.607 |
| nC6 | 0.029 |
| 22DMC5 | 0.029 |
| 24DMC5 | 0.051 |
| 223TMC4 | 0.007 |
| 33DMC5 | 0.008 |
| 2MC6 | 0.034 |
| 23DMC5 | 0.028 |
| 3MC6 | 0.023 |
| 3EtC5 | 0.001 |
| 224TMC5 | 0.404 |
| nC7 | 0.008 |
| 22DMC6 | 0.016 |
| 25DMC6 | 0.366 |
| 24DMC6 | 0.417 |
| 33DMC6 | 0.003 |
| 234TMC5 | 0.059 |
| 233TMC5 | 0.110 |
| 23DMC6 | 0.100 |
| 2M3EtC5 | 0.005 |
| 2MC7 | 0.091 |
| 4MC7 | 0.024 |
| 34DMC6 | 0.030 |
| 3MC7 | 0.071 |
| 225TMC6 | 0.048 |
| Residue | 0.178 |
| Unk C5-C8 | 0.007 |
| Total | 100.000 |
| C5+ RON | 87.0 |
| C6+ RON | 85.9 |

### EXAMPLE 10

Example 10 illustrates a process of the present invention comprising contacting isopentane and ethylene in a hydrocarbon-containing fluid in the presence of a catalyst composition under conversion conditions to provide isobutane and additional isoparaffins having from about five to about nine carbon atoms per molecule wherein the catalyst composition comprises hydrofluoric acid, sulfolane, and TiF₄.

The catalyst composition preparation and reactor system described herein in Example I was utilized. Table 19 discloses the amounts and components of the catalyst composition, the components of the hydrocarbon-containing fluid feed, and a summary product composition analysis. Table 20 discloses a detailed product composition analysis. The test data in Tables 19 and 20 clearly show that the inventive process converted over 90% of the ethylene. The data clearly demonstrate that a process of the present invention is effective in contacting isopentane and ethylene in the presence of a catalyst composition of the present invention under conversion conditions to provide at least one product hydrocarbon isomer comprising an isobutane and additional isoparaffins containing from about five to about nine carbon atoms per molecule such as 2,3-dimethylbutane, 2-methylpentane, 3-methylpentane, 2,4-dimethylpentane, 2-methylhexane, 2,3-dimethylpentane, and 3-methylhexane.

TABLE 19

| Component | Wt % |
|---|---|
| Feed | Rxn 10 |
| C2= | 6.728 |
| C2F | 0 |
| C3 | 0.002 |
| iC4 | 0.039 |
| nC4 | 0.084 |
| C4F | 0 |
| UnkC1-C4 | 0.248 |
| iC5 | 92.441 |
| nC5 | 0.455 |
| C6+ | 0.004 |
| Total | 100.000 |
| Feed wt, g | 47.9 |
| Catalyst: | |
| HF, g | 70.23 |
| TiF4, g | 22.94 |
| Sulfolane, g | 7.64 |
| Total | 100.81 |
| Mol % TiF4 | 4.9 |
| HF/S w/w | 9.19 |
| Temp, ° F. | 97.1 |
| Time, min | 30 |
| Settler Effluent Product (Summary) | |
| C2= | 0.189 |
| C2F | 0.382 |
| C3 | 0.106 |
| iC4 | 20.799 |
| nC4 | 0.371 |
| C4F | 0.000 |
| Unk C1-C5 | 0.212 |
| C5+ | 77.941 |
| Total | 100.000 |

TABLE 20
Settler Effluent Product (Detailed)

| | Rxn 10 |
|---|---|
| Component | (wt %) |
| C2= | 0.189 |
| C2F | 0.382 |
| C3 | 0.106 |
| iC4 | 20.799 |
| nC4 | 0.371 |
| Unk C1-C4 | 0.212 |
| iC5 | 32.176 |
| nC5 | 1.796 |
| 22DMC4 | 0.608 |
| 23DMC4 | 5.419 |
| 2MC5 | 13.390 |
| 3MC5 | 6.199 |
| nC6 | 0.388 |
| 22DMC5 | 0.033 |
| 24DMC5 | 2.588 |
| 223TMC4 | 0.254 |
| 33DMC5 | 0.062 |
| 2MC6 | 3.831 |
| 23DMC5 | 1.482 |
| 3MC6 | 2.733 |
| 3EtC5 | 0.114 |
| 224TMC5 | 0.049 |
| nC7 | 0.074 |
| 22DMC6 | 0.032 |
| 25DMC6 | 0.594 |
| 24DMC6 | 0.518 |
| 33DMC6 | 0.004 |
| 234TMC5 | 0.008 |
| 233TMC5 | 0.014 |
| 23DMC6 | 0.161 |
| 2M3EtC5 | 0.009 |
| 2MC7 | 0.556 |
| 4MC7 | 0.151 |
| 34DMC6 | 0.051 |
| 3MC7 | 0.445 |
| 225TMC6 | 0.578 |
| Residue | 3.498 |
| Unk C5-C8 | 0.126 |
| Total | 100.000 |
| C5+ RON | 82.6 |
| C6+ RON | 73.5 |

### EXAMPLE 11

Example 11 illustrates the effect of a lower temperature on a process of the present invention comprising contacting isopentane and ethylene in a hydrocarbon-containing fluid in the presence of a catalyst composition under conversion conditions to provide isobutane and additional reaction products comprising isoparaffins having from about five to about nine carbon atoms per molecule wherein the catalyst composition comprises hydrofluoric acid, sulfolane, and TiF₄.

The catalyst composition preparation and reactor system described herein in Example 1 was utilized. Table 21 discloses the amounts and components of the catalyst composition, the components of the hydrocarbon-containing fluid feed, and a summary product composition analysis. Table 22 discloses a detailed product composition analysis. The test data in Tables 21 and 22 clearly show that the inventive process converted over 90% of the ethylene. The data clearly demonstrate that a process of the present invention is effective in contacting isopentane and ethylene in the presence of a catalyst composition of the present invention under conversion conditions to provide at least one product hydrocarbon isomer comprising isobutane and additional isoparaffins containing from about five to about nine carbon atoms per molecule such as 2,3-dimethylbutane, 2-methylpentane, 3-methylpentane, 2,4-dimethylpentane, 2-methylhexane, 2,3-dimethylpentane, and 3-methylhexane.

TABLE 21

| Component | Wt % |
|---|---|
| Feed | Rxn 11 |
| C2= | 6.895 |
| C2F | 0 |
| C3 | 0.001 |
| iC4 | 0.038 |
| nC4 | 0.083 |
| C4F | 0 |
| UnkC1-C4 | 0.244 |
| iC5 | 92.266 |
| nC5 | 0.455 |
| C6+ | 0.017 |
| Total | 100.000 |
| Feed wt, g | 47.9 |
| Catalyst: | |
| HF, g | 70.23 |
| TiF4, g | 22.94 |
| Sulfolane, g | 7.64 |
| Total | 100.81 |
| Mol % TiF4 | 4.9 |
| HF/S w/w | 9.19 |
| Temp, ° F. | 82.0 |
| Time, min | 30 |
| Settler Effluent Product (Summary) | |
| C2= | 0.123 |
| C2F | 0.719 |
| C3 | 0.333 |
| iC4 | 6.093 |
| nC4 | 0.520 |
| C4F | 0.000 |
| Unk C1-C5 | 0.208 |
| C5+ | 92.003 |
| Total | 100.000 |

TABLE 22
Settler Effluent Product (Detailed)

| | Rxn 11 |
|---|---|
| Component | (wt %) |
| C2= | 0.123 |
| C2F | 0.719 |
| C3 | 0.332 |
| iC4 | 6.093 |
| nC4 | 0.520 |
| Unk C1-C4 | 0.208 |
| iC5 | 81.158 |
| nC5 | 0.755 |
| 22DMC4 | 0.350 |
| 23DMC4 | 0.706 |
| 2MC5 | 3.784 |
| 3MC5 | 1.734 |
| nC6 | 0.025 |
| 22DMC5 | 0.031 |
| 24DMC5 | 1.013 |
| 223TMC4 | 0.020 |
| 33DMC5 | 0.013 |
| 2MC6 | 0.311 |
| 23DMC5 | 0.562 |
| 3MC6 | 0.217 |
| 3EtC5 | 0.008 |
| 224TMC5 | 0.002 |
| nC7 | 0.006 |
| 22DMC6 | 0.008 |
| 25DMC6 | 0.043 |
| 24DMC6 | 0.037 |
| 33DMC6 | 0.002 |
| 234TMC5 | 0.000 |
| 233TMC5 | 0.000 |
| 23DMC6 | 0.011 |
| 2M3EtC5 | 0.000 |
| 2MC7 | 0.021 |
| 4MC7 | 0.005 |
| 34DMC6 | 0.003 |
| 3MC7 | 0.017 |
| 225TMC6 | 0.137 |
| Residue | 0.948 |
| Unk C5-C8 | 0.077 |
| Total | 100.000 |
| C5+ RON | 91.4 |
| C6+ RON | 76.9 |

### EXAMPLE 12

Example 12 illustrates a process of the present invention comprising contacting isobutane, normal butane, isopentane and ethylene in a hydrocarbon-containing fluid in the presence of a catalyst composition of the present invention under conversion conditions to provide at least one product hydrocarbon isomer comprising an isopentane and additional isoparaffins containing from about five to about nine carbon atoms per molecule wherein the catalyst composition comprises hydrofluoric acid, sulfolane, and TiF₄.

The catalyst composition preparation and reactor system described herein in Example 1 was utilized. Table 23 discloses the amounts and components of the catalyst composition, the components of the hydrocarbon-containing fluid feed, and a summary product composition analysis. Table 24 discloses a detailed product composition analysis. The test data in Tables 23 and 24 clearly show that the inventive process converted over 90% of the ethylene. The data clearly demonstrate that a process of the present invention is effective in contacting hydrocarbons comprising isobutane, normal butane, isopentane, and ethylene in the presence of a catalyst composition of the present invention under conversion conditions to provide at least one product hydrocarbon isomer comprising isobutane and additional isoparaffins containing from about five to about nine carbon atoms per molecule such as 2,3-dimethylbutane, 2-methylpentane, 3-methylpentane, 2,4-dimethylpentane, 2-methylhexane, 2,3-dimethylpentane, and 3-methylhexane.

TABLE 23

| Component | Wt % |
|---|---|
| Feed | Rxn 12 |
| C2= | 7.030 |
| C2F | 0 |
| C3 | 0.080 |
| iC4 | 22.339 |
| nC4 | 23.770 |
| C4F | 0 |
| UnkC1-C4 | 0.291 |
| iC5 | 46.258 |
| nC5 | 0.232 |
| C6+ | 0.001 |
| Total | 100.000 |
| Feed wt, g | 47.76 |
| Catalyst: | |
| HF, g | 69.82 |
| TiF4, g | 22.97 |
| Sulfolane, g | 7.66 |
| Total | 100.45 |
| Mol % TiF4 | 5.0 |
| HF/S w/w | 9.11 |
| Temp, ° F. | 93.8 |
| Time, min | 30 |
| Settler Effluent Product (Summary) | |
| C2= | 0.184 |
| C2F | 0.481 |
| C3 | 0.274 |
| iC4 | 27.701 |
| nC4 | 23.082 |
| C4F | 0.000 |
| Unk C1-C5 | 0.295 |
| C5+ | 47.982 |
| Total | 100.000 |

TABLE 24
Settler Effluent Product (Detailed)

| | Rxn 12 |
|---|---|
| Component | (wt %) |
| C2= | 0.184 |
| C2F | 0.481 |
| C3 | 0.274 |
| iC4 | 27.701 |
| nC4 | 23.082 |
| Unk C1-C4 | 0.295 |
| iC5 | 27.679 |
| nC5 | 1.033 |
| 22DMC4 | 0.393 |
| 23DMC4 | 2.634 |
| 2MC5 | 6.087 |
| 3MC5 | 2.794 |
| nC6 | 0.126 |
| 22DMC5 | 0.022 |
| 24DMC5 | 1.040 |
| 223TMC4 | 0.080 |
| 33DMC5 | 0.026 |
| 2MC6 | 1.223 |
| 23DMC5 | 0.578 |
| 3MC6 | 0.861 |
| 3EtC5 | 0.036 |
| 224TMC5 | 0.073 |
| nC7 | 0.020 |
| 22DMC6 | 0.014 |
| 25DMC6 | 0.252 |
| 24DMC6 | 0.231 |
| 33DMC6 | 0.002 |
| 234TMC5 | 0.012 |
| 233TMC5 | 0.022 |
| 23DMC6 | 0.067 |
| 2M3EtC5 | 0.004 |
| 2MC7 | 0.157 |
| 4MC7 | 0.042 |
| 34DMC6 | 0.020 |
| 3MC7 | 0.124 |
| 225TMC6 | 0.587 |
| Residue | 1.674 |
| Unk C5-C8 | 0.071 |
| Total | 100.000 |
| C5+ RON | 86.6 |
| C6+ RON | 76.0 |

### EXAMPLE 13

Example 13 illustrates a process of the present invention comprising contacting normal pentane and ethylene in a hydrocarbon-containing fluid in the presence of a catalyst composition under conversion conditions to provide at least one product hydrocarbon isomer comprising isobutane and additional reaction products comprising isoparaffins having from about five to about nine carbon atoms per molecule wherein the catalyst composition comprises hydrofluoric acid, sulfolane, and TiF₄.

The catalyst composition preparation and reactor system described herein in Example 1 was utilized. Table 25 discloses the amounts and components of the catalyst composition, the components of the hydrocarbon-containing fluid feed, and a summary product composition analysis. Table 26 discloses a detailed product composition analysis. The test data in Tables 25 and 26 clearly show that the inventive process converted over 90% of the ethylene. The data clearly demonstrate that a process of the present invention comprises contacting normal pentane and ethylene in the presence of a catalyst composition of the present invention under conversion conditions to provide at least one product hydrocarbon isomer comprising an isobutane and additional isoparaffins containing from about five to about nine carbon atoms per molecule such as isopentane, 2,3-dimethylbutane, 2-methylpentane, 3-methylpentane, 2,4-dimethylpentane, 2-methylhexane, and 3-methylhexane.

TABLE 25

| Component | Wt % |
|---|---|
| Feed | Rxn 13 |
| C2= | 6.625 |
| C2F | 0 |
| C3 | 0.003 |
| iC4 | 0.000 |
| nC4 | 0.000 |
| C4F | 0 |
| UnkC1-C4 | 0.103 |
| iC5 | 0.505 |
| nC5 | 92.698 |
| C6+ | 0.066 |
| Total | 100.000 |
| Feed wt, g | 47.73 |
| Catalyst: | |
| HF, g | 70.01 |
| TiF4, g | 22.95 |
| Sulfolane, g | 7.62 |
| Total | 100.58 |
| Mol % TiF4 | 4.9 |
| HF/S w/w | 9.19 |
| Temp, ° F. | 94.6 |
| Time, min | 30 |
| Settler Effluent Product (Summary) | |
| C2= | 0.084 |
| C2F | 0.399 |
| C3 | 0.119 |
| iC4 | 4.352 |
| nC4 | 0.298 |
| C4F | 0.000 |
| Unk C1-C5 | 0.010 |
| C5+ | 94.737 |
| Total | 100.000 |

TABLE 26
Settler Effluent Product (Detailed)

| | Rxn 13 |
|---|---|
| Component | (wt %) |
| C2= | 0.084 |
| C2F | 0.399 |
| C3 | 0.119 |
| iC4 | 4.352 |
| nC4 | 0.298 |
| Unk C1-C4 | 0.010 |
| iC5 | 4.619 |
| nC5 | 81.576 |
| 22DMC4 | 0.217 |
| 23DMC4 | 0.742 |
| 2MC5 | 1.645 |
| 3MC5 | 0.756 |
| nC6 | 0.104 |
| 22DMC5 | 0.020 |
| 24DMC5 | 0.478 |
| 223TMC4 | 0.152 |
| 33DMC5 | 0.024 |
| 2MC6 | 0.674 |
| 23DMC5 | 0.272 |
| 3MC6 | 0.478 |
| 3EtC5 | 0.020 |
| 224TMC5 | 0.020 |
| nC7 | 0.037 |
| 22DMC6 | 0.033 |
| 25DMC6 | 0.215 |
| 24DMC6 | 0.187 |
| 33DMC6 | 0.004 |
| 234TMC5 | 0.003 |
| 233TMC5 | 0.006 |
| 23DMC6 | 0.057 |
| 2M3EtC5 | 0.003 |
| 2MC7 | 0.206 |
| 4MC7 | 0.056 |
| 34DMC6 | 0.018 |
| 3MC7 | 0.164 |
| 225TMC6 | 0.176 |
| Residue | 1.654 |
| Unk C5-C8 | 0.118 |
| Total | 100.000 |
| C5+ RON | 64.0 |
| C6+ RON | 71.5 |

The results shown in the above examples clearly demonstrate that the present invention is well adapted to carry out the objects and attain the ends and advantages mentioned as well as those inherent therein.

Reasonable variations, modifications, and adaptations can be made within the scope of the disclosure and the appended claims without departing from the scope of this invention.

That which is claimed:

1. A process comprising contacting at least one feed hydrocarbon selected from the group consisting of paraffins, isoparaffins, and combinations thereof comprising from three to about seven carbons atoms per molecule and ethylene in the presence of a catalyst composition under conversion conditions to provide at least one product hydrocarbon isomer comprising an isoparaffin comprising from about four to about nine carbon atoms per molecule wherein said catalyst composition comprises a hydrogen halide component, a sulfone component, and a metal halide component.

2. A process according to claim 1 wherein said paraffins are selected from the group consisting of propane, butane, pentane, hexane, heptane and combinations thereof.

3. A process according to claim 1 wherein said isoparaffins are selected from the group consisting of isobutane, isopentane, 2,2-dimethylbutane, 2,3-dimethylbutane, 2-methylpentane, 3-methylpentane, 2,2-dimethylpentane, 2,4-dimethylpentane, 2,2,3-trimethylbutane, 3,3-dimethylpentane, 2-methylhexane, 2,3-dimethylpentane, 3-methylhexane, 3-ethylpentane, and combinations thereof.

4. A process according to claim 1 wherein said at least one product hydrocarbon isomer is selected from the group consisting of isobutane, isopentane, 2,2-dimethylbutane, 2,3-dimethylbutane, 2-methylpentane, 3-methylpentane, 2,2-dimethylpentane, 2,4-dimethylpentane, 2,2,3-trimethylbutane, 3,3-dimethylpentane, 2-methylhexane, 2,3-dimethylpentane, 3-methylhexane, 3-ethylpentane, 2,2,4-trimethylpentane, 2,2-dimethylhexane, 2,5-dimethylhexane, 2,4-dimethylhexane, 3,3-dimethylhexane, 2,3,4-trimethylpentane, 2,3,3-trimethylpentane, 2,3-dimethylhexane, 2-methyl-3-ethylpentane, 2-methylheptane, 4-methylheptane, 3,4-dimethylhexane, 3-methylheptane, 2,2,5-trimethylhexane, and combinations thereof.

5. A process according to claim 1 wherein said at least one feed hydrocarbon comprises butane and said at least one product hydrocarbon isomer comprises isobutane.

6. A process according to claim 5 wherein said at least one product hydrocarbon isomer further comprises isopentane, 2,2-dimethylbutane, 2,3-dimethylbutane, 2-methylpentane, 3-methylpetane, 2,2-dimethylpentane, 2,4-dimethylpentane, 2-methylhexane, 3-methylhexane, and 2,2-dimethylhexane.

7. A process according to claim 1 wherein said at least one feed hydrocarbon comprises isobutane and said at least one product hydrocarbon isomer comprises isopentane.

8. A process according to claim 7 wherein said at least one product hydrocarbon isomer further comprises 2,2-dimethylbutane, 2,3-dimethylbutane, 2-methylpentane, 3-methylpetane, 2,2,4-trimethylpentane, 2,5-dimethylhexane, and 2,4-dimethylhexane.

9. A process according to claim 1 wherein said at least one feed hydrocarbon comprises propane and said at least one product hydrocarbon isomer comprises isobutane.

10. A process according to claim 9 wherein said at least one product hydrocarbon isomer further comprises isopentane, 2-methylpentane, and 2-methylhexane.

11. A process according to claim 1 wherein said at least one feed hydrocarbon comprises hydrocarbons comprising six or more carbon atoms per molecule and said at least one product hydrocarbon isomer comprises isobutane.

12. A process according to claim 11 wherein said at least one product hydrocarbon isomer further comprises 2-methylhexane, 3-methylhexane, and 2,2-dimethylhexane.

13. A process according to claim 1 wherein said at least one feed hydrocarbon comprises isopentane and said at least one product hydrocarbon isomer comprises isobutane.

14. A process according to claim 13 wherein said at least one product hydrocarbon isomer further comprises 2,3-dimethylbutane, 2-methylpentane, 3-methylpentane, 2,4-dimethylpentane, 2-methylhexane, 2,3-dimethylpentane, and 3-methylhexane.

15. A process according to claim 1 wherein said at least one feed hydrocarbon comprises isobutane and butane and said at least one product hydrocarbon isomer comprises isopentane.

16. A process according to claim 15 wherein said at least one product hydrocarbon isomer further comprises 2,3-dimethylbutane, 2-methylpentane, 3-methylpentane, 2,5-dimethylhexane, and 2,4-dimethylhexane.

17. A process according to claim 1 wherein said at least one feed hydrocarbon comprises isobutane, butane, and isopentane and said at least one product hydrocarbon isomer comprises isobutane.

18. A process according to claim 17 wherein said at least one product hydrocarbon isomer further comprises 2,3-dimethylbutane, 2-methylpentane, 3-methylpentane, 2,4-dimethylpentane, 2-methylhexane, 2,3-dimethylpentane, and 3-methylhexane.

19. A process according to claim 1 wherein said at least one feed hydrocarbon comprises pentane and said at least one product hydrocarbon isomer comprises isobutane.

20. A process according to claim 19 wherein said at least one product hydrocarbon isomer further comprises isopentane, 2,3-dimethylbutane, 2-methylpentane, 3-methylpentane, 2,4-dimethylpentane, 2-methylhexane, and 3-methylhexane.

21. A process according to claim 1 wherein said hydrogen halide component is selected from the group consisting of hydrogen fluoride, hydrogen chloride, hydrogen bromide, and combinations thereof.

22. A process according to claim 21 wherein said hydrogen halide component is hydrogen fluoride.

23. A process according to claim 1 wherein said sulfone component comprises a sulfone of the general formula R—SO₂—R′ wherein R and R′ are monovalent hydrocarbon alkyl or aryl substituents containing from 1 to 8 carbon atoms.

24. A process according to claim 23 wherein said sulfone component is selected from the group consisting of sulfolane, 3-methylsulfolane, 2,4-dimethylsulfolane, and combinations thereof.

25. A process according to claim 24 wherein said sulfone component is sulfolane.

26. A process according to claim 1 wherein a metal of said metal halide component is selected from the group consisting of the metals of Groups III, IV and V of the Periodic Table of Elements.

27. A process according to claim 26 wherein said metal of said metal halide component is selected from the group consisting of B, Al, Ga, In, Sn, Ti, Zr, P, As, Sb, Bi, V, Nb, Ta, and combinations thereof.

28. A process according to claim 27 wherein a halide of said metal halide component is selected from the group consisting of fluoride, bromide, chloride, and combinations thereof.

29. A process according to claim 28 wherein said metal halide component is selected from the group consisting of SbF₅, TaF₅, PF₅, NbF₅, BF₃, SnF₄, TiF₄, AlCl₃, SnCl₄, AlBr₃, and combinations thereof.

30. A process according to claim 29 wherein said metal halide component is TiF₄.

31. A process according to claim 1 wherein said conversion conditions comprise:
a temperature of at least about 0° F.;
a temperature of no more than about 250° F.;
a pressure of at least about 40 psig;
a pressure of no more than about 1000 psig;
a time period of at least about 0.05 minute; and
a time period of more than about 2 hours.

32. A process according to claim 1 wherein said at least one feed hydrocarbon and said ethylene are present in a hydrocarbon-containing fluid.

33. A process according to claim 1 wherein a weight ratio of total hydrocarbon to ethylene is at least about 1:1 and no more than about 30:1.

34. A process according to claim 1 wherein a weight percent of said hydrogen halide component based on the total weight of said catalyst composition is at least about 50 and no more than about 90, a weight percent of said sulfone component based on the total weight of said catalyst composition is at least about 10 and no more than about 35, and a weight percent of said metal halide component based on the total weight of said catalyst composition is at least about 0.01 and no more than about 20.

35. A process according to claim 1 wherein a conversion of said ethylene is at least about 50 weight percent.

36. A process according to claim 1 wherein a conversion of said ethylene is at least about 80 weight percent.

37. A process according to claim 1 wherein a weight ratio of said catalyst composition to total hydrocarbon and ethylene is at least about 0.5:1 and no more than about 20:1.

38. A process according to claim 1 wherein said catalyst composition comprises hydrofluoric acid, sulfolane, and TiF₄.

39. A process according to claim 1 wherein said contacting comprises converting said at least one feed hydrocarbon.

40. A process according to claim 1 wherein said converting is selected from the group consisting of reacting, alkylating, isomerizing, disproportionating, and combinations thereof.
